(19) United States
(12) Patent Application Publication
(10) Pub. No.: US 2024/0065697 A1
(43) Pub. Date: Feb. 29, 2024

(54) SYSTEMS AND METHODS TO INCREASE IMPLANTABLE DEVICE VOLUME FOR TREATMENT OF ANEURYSMS

(71) Applicant: DePuy Synthes Products, Inc., Raynham, MA (US)

(72) Inventors: Emilie KOTTENMEIER PARK, Los Angeles, CA (US); Alia KHALED, Burlington (CA); Patrick BROUWER, Haarlem (NL)

(73) Assignee: DePuy Synthes Products, Inc., Raynham, MA (US)

(21) Appl. No.: 18/233,007

(22) Filed: Aug. 11, 2023

Related U.S. Application Data
(60) Provisional application No. 63/401,940, filed on Aug. 29, 2022.
(60) Provisional application No. 63/425,413, filed on Nov. 15, 2022.

(51) Int. Cl. A61B 17/12

(52) U.S. Cl. CPC A61B 17/12113; A61B 17/1214

(57) ABSTRACT

A method for treating an aneurysm by delivering, to an aneurysm sac, an occlusion device comprising a coiled implant releasably retained on the occlusion device. The coiled implant comprises an outer diameter that increases a coil volume in the aneurysm compared to comparative devices having approximately equal lengths and varied primary wind diameter, or comparative devices having approximately equal coil widths and varied lengths.

### CROSS-REFERENCE TO RELATED APPLICATIONS

This application claims the benefit of U.S. Provisional Patent Application No. 63/401,940, filed Aug. 29, 2022, which is incorporated by reference herein in its entirety.

This application also claims the benefit of U.S. Provisional Patent Application No. 63/425,413, filed Nov. 15, 2022, which is incorporated by reference herein in its entirety.

### FIELD

The present invention generally relates to implantable medical devices and methods which are used to occlude vessels within a patient, and more particularly to embolic coils having varying dimensions.

### BACKGROUND

Aneurysms can be intravascularly treated by delivering a treatment device to the aneurysm to fill the sac of the aneurysm with embolic material and/or block the neck of the aneurysm to inhibit blood flow into the aneurysm. When filling the aneurysm sac, the embolic material can promote blood clotting to create a thrombotic mass within the aneurysm. When treating the aneurysm neck without substantially filling the aneurysm sac, blood flow into the neck of the aneurysm can be inhibited to induce venous stasis in the aneurysm and facilitate natural formation of a thrombotic mass within the aneurysm.

In some current treatments, multiple metal-based (e.g. platinum) embolic coils are used to either fill the aneurysm sac or treat the entrance of the aneurysm neck. When the aneurysm sac is packed with embolic coils, the aneurysm typically takes on the shape of the metal coils packed within it, making the aneurysm a permanent volume within the skull. Placing embolic coils exclusively at the aneurysm neck can allow thrombus to form naturally within the aneurysm sac and allow the sac to shrink in volume. In some instances, when either the aneurysm sac is packed with embolic coils or the neck is treated, blood flow can resume into the aneurysm following an initial treatment, requiring a subsequent treatment where additional embolic coils or treatment devices are placed to treat the aneurysm, further increasing the volume of metal at the aneurysm, and possibly extending the aneurysm sac.

Typically, it is desired to deploy enough coils to obtain a packing density of about 20% or more, preferably about 35% and more if possible. As the number of coiling procedures for intracranial aneurysms (IAs) performed increases, total procedure costs may become an important consideration for hospitals. The cost of each embolic coil and the number of coils used to treat an IA drive total material costs for procedures. Certain dimensions of the embolic coils may have an influence on the total material cost as well as patient outcomes.

### SUMMARY

The subject of this disclosure is the use of an occlusion device to treat an aneurysm from the neurovascular vessels. An example method for treating an aneurysm can include delivering, to an aneurysm sac, an occlusion device having a coiled implant releasably retained on the occlusion device, releasing the coiled implant in the aneurysm sac, and achieving, by the coiled implant, an increase in packing density in the aneurysm sac compared to a comparative device having an approximately equal coil length of the coiled implant.

An example method for treating an aneurysm can include delivering, to an aneurysm sac, a coiled implant releasably retained on an occlusion device, positioning the coiled implant in the aneurysm sac, and achieving, by the coiled implant, an increase in coil volume in the aneurysm sac compared to a comparative device having an approximately equal coil width of the coiled implant.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 illustrates a cut-away view of an example implant according to aspects of the present invention.

FIG. 2A illustrates a cross-sectional view of the example implant illustrated in FIG. 1.

FIG. 2B illustrates a portion of an example implant according to aspects of the present invention.

FIGS. 3 through 5 illustrate example implant with varying softness and stiffness according to aspects of the present invention.

FIG. 6A is a table of example implants and comparative devices of equivalent lengths with varying PWDs, according to aspects of the present invention.

FIG. 6B is a table of example implants and comparative devices of varying lengths and varying PWDs, according to aspects of the present invention.

FIG. 7A illustrates formation of an outer perimeter arrangement within an aneurysm according to aspects of the present invention.

FIG. 7B illustrates formation of an interior array within the outer perimeter arrangement of FIG. 7A according to aspects of the present invention.

FIG. 8 is a flowchart of a method of treating an aneurysm, according to aspects of the present invention.

FIG. 9 is a flowchart of a method of treating an aneurysm, according to aspects of the present invention.

### DETAILED DESCRIPTION

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. By “comprising” or “containing” or “including” it is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As discussed herein, vasculature of a “subject” or “patient” may be vasculature of a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to a human (e.g., rat, dog, pig, monkey, or the like). It should be appreciated that the subject may be any applicable human patient, for example.

As discussed herein, “operator” may include a doctor, surgeon, or any other individual or delivery instrumentation associated with delivery of a clot retrieval device to the vasculature of a subject.

As discussed herein, “aneurysm” or “aneurysm sac” can be understood as a bulge or ballooning of a wall of a blood vessel in the circulatory system, generally the brain. Typically, an aneurysm develops against the weakened wall of an arterial blood vessel. The force of the blood pressure against the weakened wall causes the wall to abnormally bulge or balloon outwardly.

Intravascular catheter techniques typically involve using a catheter to deliver an occlusion device (e.g., embolic coils) to a preselected location within the vasculature of a patient. For example, in the case of a cranial aneurysm, methods and procedures which are well known in the art are used for inserting and guiding the distal end of a delivery catheter into the vasculature of a patient to the site of the cranial aneurysm.

Once the occlusion device has been delivered to and deployed into the aneurysm, the blood within the aneurysm will generally clot in and around the occlusion device to form a thrombus. The terms, “clot”, “thrombus”, “obstruction”, “occlusion”, “blockage”, and/or the like, can be and are often used interchangeably throughout this disclosure.

“Occlusion devices” can include, but not be limited to, one or more coils, releasable wires, meshes, balloons, embolic devices, one or more combinations thereof, and/or the like, each of which are often used interchangeably throughout this disclosure. Embolic coils are typically constructed from a metal wire which may be wound into a variety of shapes, including a helical shape. Delivery of an “occlusion device” is typically accomplished via delivery of one or more catheters into the femoral artery and/or the radial artery, guided into the arteries of the brain, vascular bypass, angioplasty, and/or the like.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g., “about 90%” may refer to the range of values from 71% to 99%.

As used herein, “packing density” is the ratio of the coil volume to the total volume of the aneurysm.

Examples presented herein generally include placing an implant into an aneurysm sac. The implant can have a coiled shape similar to a metallic embolic coil. A potential advantage of the coiled shape is that in some examples, the coiled implant can be delivered using delivery systems suitable for delivering an embolic coil as understood by a person skilled in the pertinent art.

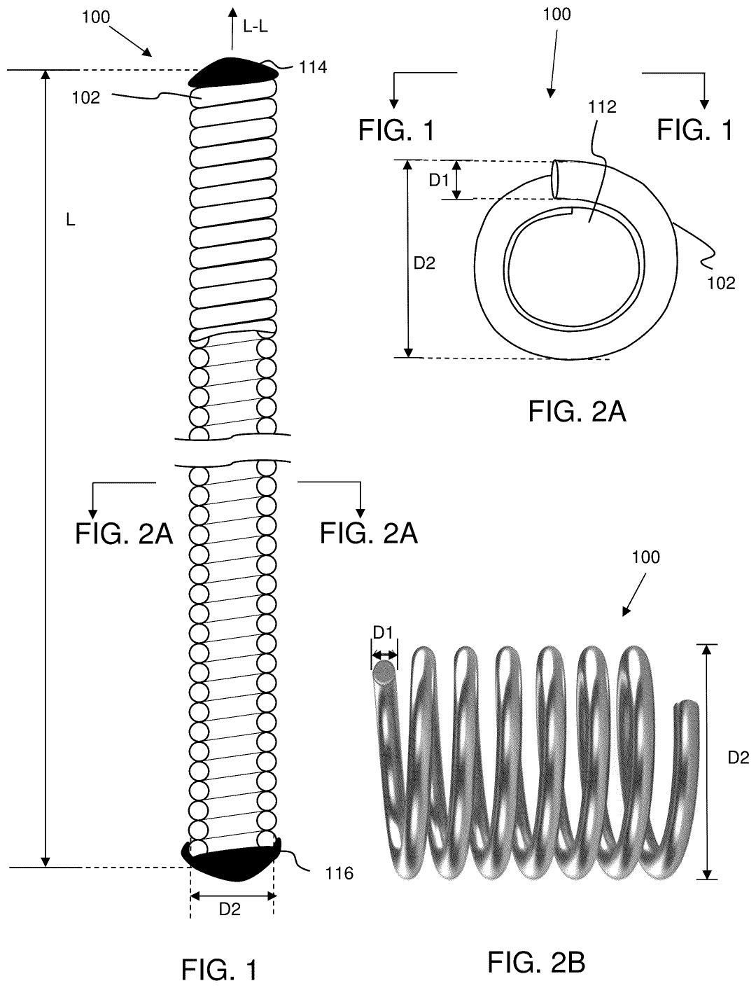

FIG. 1 illustrates a cut-away view of an example occlusion device 100. Although not depicted in FIG. 1, the occlusion device 100 can be delivered from a delivery catheter and connected to a vascular occlusion delivery system. The delivery catheter is a typical catheter used for neurovascular procedures. The catheter size is selected in consideration of the size, shape, and directionality of the aneurysm or the body lumens the catheter must pass through to get to the treatment site. The delivery catheter may have a total usable length anywhere from 80 centimeters to 165 centimeters and a distal length of anywhere between 5 centimeters to 42 centimeters. The delivery catheter may have an inner diameter (ID) of anywhere between 0.010 and 0.030 inches. The outer diameter (OD) may also range in size and may narrow at either its proximal section or distal section. The outer diameter may be 3 French or less. For the below examples, the proximal section of an inner embolic device is the end closet to the physician and the distal section is farthest away from the physician.

An occlusion device 100 typically exits a distal section of the delivery catheter to be deployed into an aneurysm. A proximal section of the delivery catheter can house the delivery system. The delivery system is typically removably connected to a proximal section 102 of the occlusion device 100 to deploy and/or retrieve the occlusion device 100 out of the delivery catheter and into the aneurysm. Delivery systems are known to those of skill in the art and any can be used with any example of the present invention to deploy and/or retrieve coils, meshes, or other devices. Delivery systems may include pusher members with any of the known mechanisms to release the vascular occlusion device 100, which can include mechanical, electrical, hydraulic, or thermal mechanisms. In some examples, the vascular occlusion device 100 is pushed out of the delivery catheter and into the aneurysm, as opposed to placing the delivery catheter and device 100 in the aneurysm and removing the catheter.

The occlusion device 100 includes a coiled implant 102 having an outer diameter D2 of the coiled shape of the coiled implant 102. The coiled implant 102 extends along a longitudinal axis L-L and has a length L measured between a proximal end 116 and a distal end 114 of the coiled implant 102. When an aneurysm sac is packed with coiled implants 102, the length L can vary among delivered implants such that the volume of the aneurysm is filled to achieve an optimal packing density.

As illustrated, the coiled implant 102 can be capped at the proximal end 116 and the distal end 114. The distal end 114 can be bare or have an atraumatic cap as understood by a person skilled in the pertinent art. The proximal end 116 can include a detachment feature configured to interface with an implant delivery system, for example a delivery system suitable for delivering a metallic embolic coil, a variation thereof, or an alternative thereto as understood by a person skilled in the pertinent art.

In some embodiments, the implant can include crossing microfibers to initiate thrombosis within the aneurysm sac. Another potential advantage of the coiled shape is that crossing microfibers may be held between windings of the coiled implant by interference fit or friction fit. The implant can include a stretch-resistant member extending through a lumen of the coiled shape to inhibit elongation of the coiled shape. The crossing microfibers can be attached to the coiled implant by friction fit between coil windings, heat bonding to the coiled implant, and/or securement to the stretch-resistant member.

The implant can have an absorption rate long enough so that the aneurysm can become sufficiently occluded and sufficiently healed as the implant is absorbed. The coiled implant, crossing microfibers, and stretch-resistant member can have rates of absorption that differ to promote an accelerated sequence of occlusion and healing of the aneurysm.

Bioabsorbable materials suitable for the implant can include, but are not limited to polyethylbenzene, polydimethylsiloxane, polyglycolic acid, poly-L-lactic acid, polycaprolactive, polyhydroxybutyrate, polyhydroxyvalerate, polydioxanone, polycarbonate, polyanhydride, polycaprolactone, polydioxanone, polybutyrolactone, polyvalerolactone, poly(lactic-co-glycolic acid), cellulose acetate propionate, and combinations thereof. Material composition of component parts of the implant can be selected to achieve a desired absorption rate/time for the respective component parts. Geometry can also affect absorption rate/time such that two component parts having the same material composition and differing geometry can be absorbed at different absorption rates/times. For instance, when the crossing microfibers and the coiled implant have identical material compositions, the crossing microfibers may be absorbed in a shorter absorption time compared to the coiled implant due to a larger ratio of surface area to volume of the crossing microfibers.

FIG. 2A illustrates a cross-sectional view of the example occlusion device 100 having a coiled implant 102 as indicated in FIG. 1. The coiled implant 102 can have a coil width (D1) from about 0.001 inches (about 25 micrometers) to about 0.003 inches (about 76 micrometers). The outer diameter D2, or primary wind diameter (PWD), of the coiled configuration of the coiled implant 102 can measure from about 0.008 inches (about 200 micrometers) to about 0.018 inches (about 460 micrometers), or more preferably from about 0.010 inches to about 0.012 inches.

A coil's volume is directly related to its length from end-to-end and its primary wind diameter (PWD), as shown in more detail in FIG. 2B. The effect of coil length on cost efficiency has been demonstrated; however, studies that assess the relationship between coil PWD and cost efficiency are lacking. This study uses a hypothetical aneurysm model and a case study to evaluate the influence of PWD on packing density, the number of coils required to achieve a desired packing density, and the economic impact of PWD.

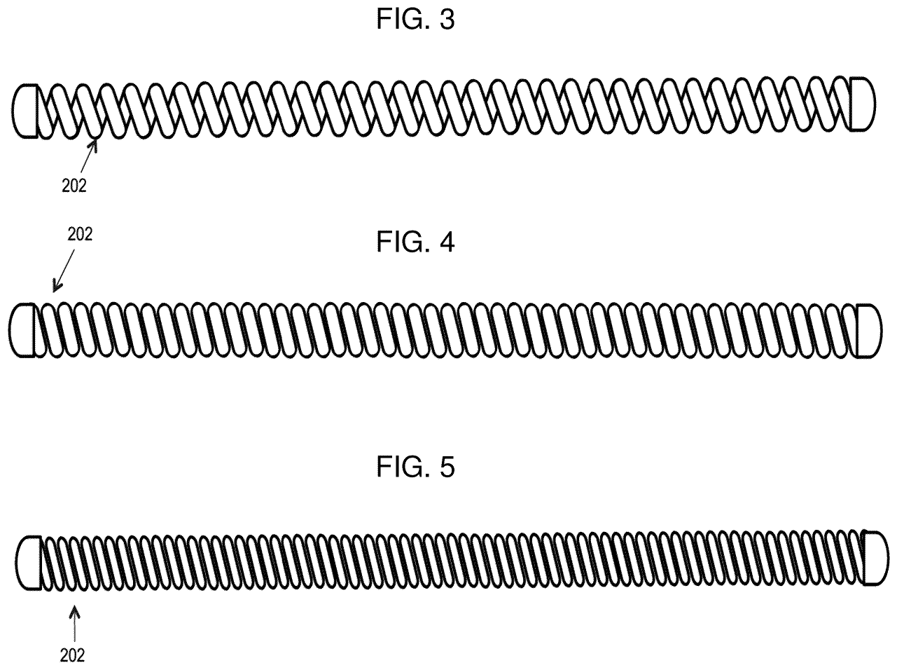

A coil can vary along its length in softness and in stiffness. FIGS. 3-5 illustrate different example coil implants 202 with varying stiffness. This can be by opening the coil pitch or primary wind diameter (FIG. 3), increasing the coil diameter to change the coil width (D1) from a larger wire (FIG. 4) to a smaller wire (FIG. 5). Additionally, a coil can be annealed in sections to soften the metal.

To analyze the effect of PWD on coil volume and subsequent packing density, a hypothetical aneurysm model was employed. The hypothetical aneurysms were filled with a set of embolic coils, and the resulting packing density was determined by first calculating the aneurysm volume and total sum of coil volumes. The volume of the hypothetical spherical aneurysm was calculated using the formula for the volume of a sphere (4/3πr³).

To assess the impact of PWD on packing density, an analysis was conducted using a set of coils that differed only by their PWD (i.e., cost per coil, coil lengths, and coil shape were the same). Coils in this analysis had a PWD of either 0.010″ or 0.012″, and all coils were complex in shape. Coils with 0.010″ PWD were selected as a comparative coil. The cost per coil used was $1,429, based on the mean cost of coils (in 2015-2016 U.S. dollars) described in a previous study, and a scenario analysis was conducted using a range of coil costs from other published literature sources ($1,295-1,695). See Gandhoke G S. Pandya Y K, Jadhav A P, et al. Cost of coils for intracranial aneurysms: clinical decision analysis for implementation of a capitation model. J. Neurosurg. 2018: 128:1792-1798; see also Milburn J., Pansara. A L, Vidal G, et al. Initial experience using the Penumbra coil 400: comparison of aneurysm packing, cost effectiveness, and coil efficiency. J. Neurointery Surg. 2014; 6:121-124. The formula used to calculate coil volume was [(π)(PWD/2)²(length)], and packing density was calculated by [(E coil volumes/aneurysm volume)(100%)].

In any of the embodiments disclosed herein, occlusion device 100 can include a coil from the CEREPAK Detachable Coil System WERENOVUS, Miami, Florida), including the Freeform Microcoil. The Freeform Minim Microcoil. The Freeform Xtrasoft MiCrOCOil, The Unithrm. Microcoil, The Uniform XL Microcoil, The Helifonn XL Microcoil, And/Or The Heliform Xtrasoft XL Microcoil. Comparative devices can include Stryker Target detachable coils (STRYKER NEUROVASCULAR, Fremont, California) of varying shapes and sizes, including the Target 360, Target XL, Target 3D, and Target Nano.

As disclosed herein, the influence of PWD (D2) and length (L) was analyzed using the Coil Volume Formula: [π(D2/2)² (L)]. Relative % difference in total coil volume was compared between four comparative coil devices with four matched occlusion device 100 coils in two scenarios. First, the coil volume for identical length and varying PWD was calculated. Second, both PWD and length were varied.

Results:

Comparative device 1 having dimensions of 10 mm×40 cm (D2=0.014″, length=40 cm) with volume of 39.726 mm³ was matched to occlusion device 100a (CEREPAK Heliform XL) having dimensions of 10 mm×40 cm (D2=0.015″, length=40 cm) with 15% greater volume of 45.604 mm³.

Comparative device 2 having dimensions 3 mm×6 cm (D2=0.010″, length=6 cm) with volume 3.040 mm³ was matched to occlusion device 100b (CEREPAK Freeform) having dimensions 3 mm×6 cm (D2=0.012″, length=6 cm) with 44% greater volume of 4.378 mm³.

Comparative device 3 having dimensions 12 mm×45 cm (D2=0.014″, length=45 cm) with volume 44.692 mm³ was matched to occlusion device 100c (CEREPAK Heliform XL) having dimensions 12 mm×42 cm (D2=0.015″, length=42 cm) with 7% greater volume of 47.884 mm³.

Comparative device 4 having dimensions 5 mm×20 cm (D2=0.010″, length=20 cm) with volume 10.134 mm³ was matched to occlusion device 100d (CEREPAK Freeform) having dimensions 5 mm×15 cm (D2=0.012″, length=l5 cm) with 8% greater volume of 10.945 mm³.

Conclusions:

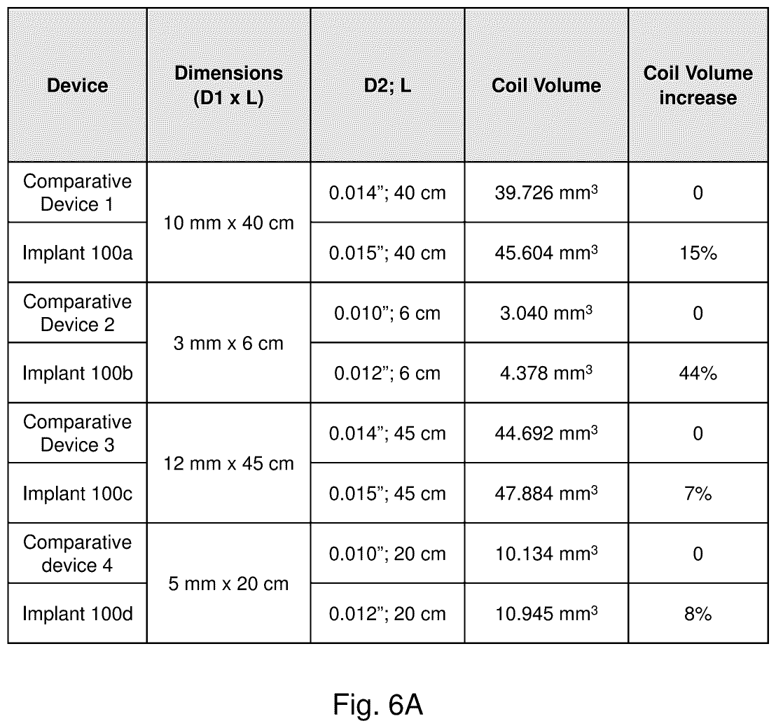

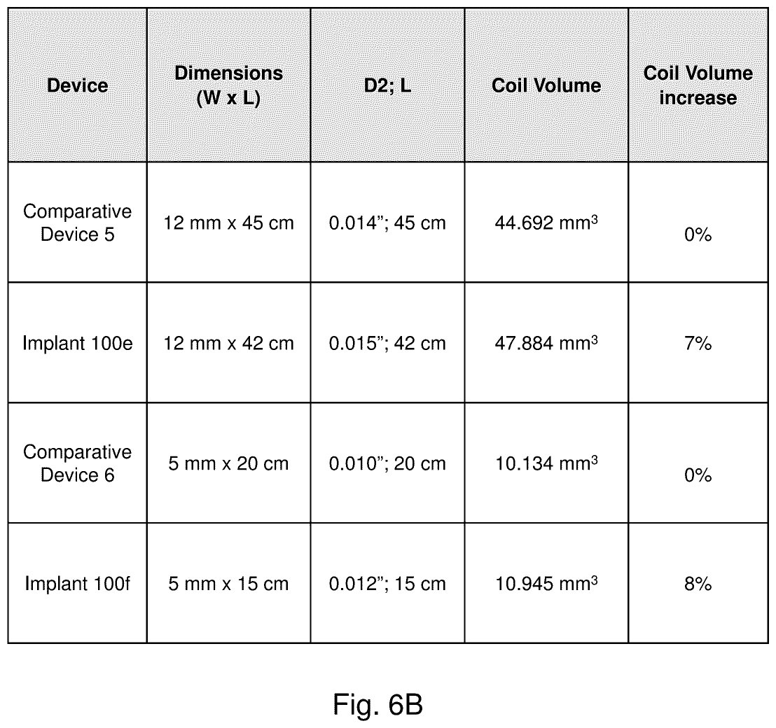

As shown in the table of FIG. 6A, with equivalent length, an increase in D2 (by as little as 0.001″-0.002″) can impact relative coil volume by 15%-44%. Shorter coils (by 3-5 cm) with D2=0.012″ vs. 0.010″ have slightly greater coil volume ranging from 7%-8%. Clinicians evaluate many characteristics when treating an intracranial aneurysm; however, D2 might be given consideration over length due to its impact on total coil volume. Occlusion device 100a-100d coils contain a higher metal volume when compared to the most similar comparative device 1-4 counterparts.

As shown in the table of FIG. 6B, with varying length, an increase in D2 (by as little as 0.001″-0.002″) can impact relative coil volume by 7%-8%, even with longer length coils of comparative devices 5 and 6 compared to implants 100e and 100f, respectively (varying coil length by 3-5 cm). In particular, when occlusion device 100e has a shorter coil length (by 3 cm) but a larger diameter (D2=0.015″ vs. 0.014″), occlusion device 100e provides an increased coil volume of about 7% compared to comparative device 5. Similarly, occlusion device 100f has a shorter coil length (by 5 cm) but a larger diameter (D2=0.012″ vs. 0.010″) and increases the coil volume by approximately 8% compared to comparative device 6. As discussed supra, D2 might be given consideration over length or width due to its impact on total coil volume. Implants 100e and 100f coils contain a higher metal volume when compared to the most similar comparative device 5 and 6 counterparts.

In some embodiments, increasing D2 may lead to greater volumetric and cost efficiency in delivering occlusion device 100 due to a decrease in number of coiled implants 102 needed per procedure, compared to a comparative device having similar coil lengths and coil widths.

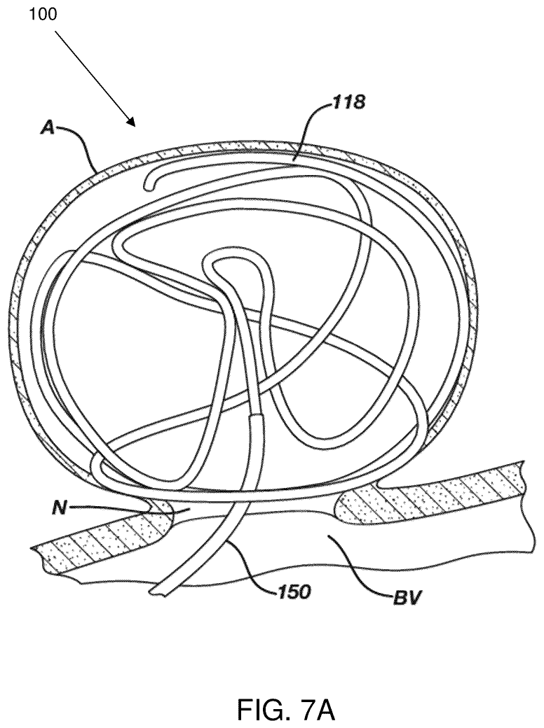

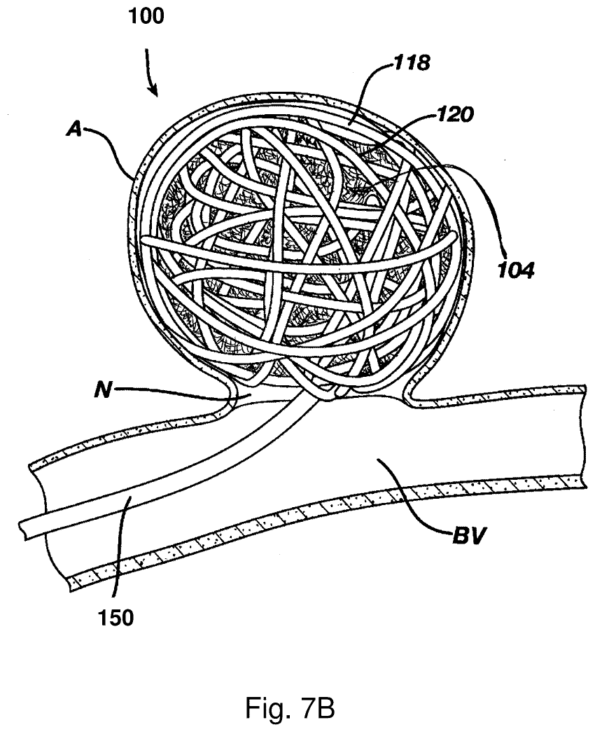

FIGS. 7A and 7B illustrate treatment of an aneurysm A with an example occlusion device configured similarly to the occlusion device 100 disclosed herein or comparative device. A delivery tube 150 can be positioned at the neck N of the aneurysm A and/or within the sac of the aneurysm A and the occlusion device 100 can be moved distally through the delivery tube 150 into the aneurysm sac. FIG. 7A illustrates formation of an outer perimeter arrangement 118 within an aneurysm A. A distal portion of the occlusion device 100 winds within the aneurysm sac as the occlusion device 100 is moved distally out of the delivery tube 150 so that the coiled implant 102 forms a crisscrossing network at the wall of the aneurysm A and across the neck N of the aneurysm A. As density of coiled implant 102 crisscrossed across the neck N of the aneurysm A increases, the perimeter arrangement 118 forms a barrier across the aneurysm neck N inhibiting the proximal portion 106 of the occlusion device 100 from exiting the aneurysm sac.

Additionally, or alternatively, a first implant can be implanted to form the outer perimeter arrangement 118 illustrated in FIG. 7A. The outer perimeter arrangement 118 may form a barrier across the neck N of the aneurysm as understood by a person skilled in the pertinent art. The occlusion device 100, as illustrated in FIG. 1, can have a coil width (W) from about 0.001 inches (about 25 micrometers) to about 0.003 inches (about 76 micrometers). The outer diameter D2, or primary wind diameter (PWD), of the coiled configuration of the coiled implant 102 can measure from about 0.008 inches (about 200 micrometers) to about 0.018 inches (about 460 micrometers). The length L of the coiled implant 102 can follow the outer perimeter and form an interior array 120 similar to the coils as illustrated in FIG. 7B.

Although coil volume is an input for packing density, it was not understood in the industry that a change in PWD by even 0.001″ or 0.002″ could impact the coil volume more so than length impacts coil volume, as demonstrated in FIGS. 6A and 6B.

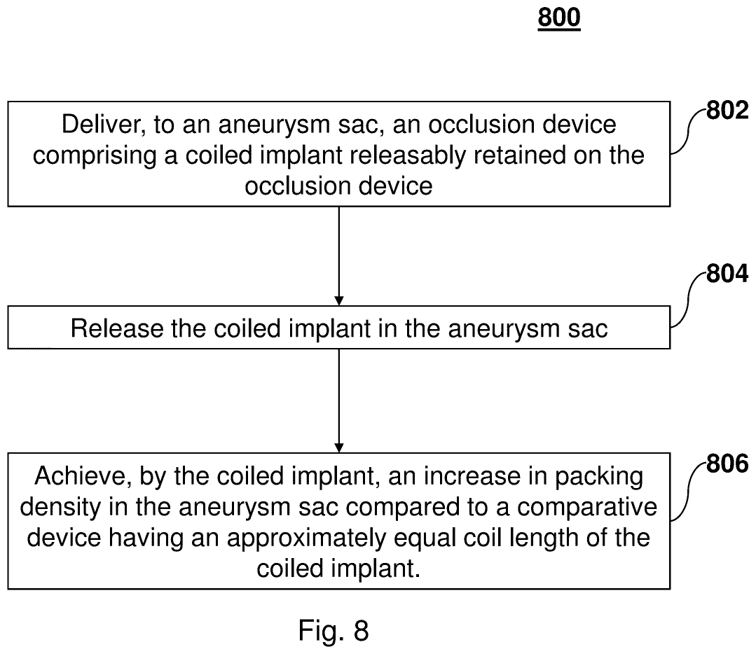

FIG. 8 is a flowchart of a method 800 of treating an aneurism. Method 800 can include delivering, to an aneurysm sac, an occlusion device 100 comprising a coiled implant 102 releasably retained on the occlusion device 100 at step 802. Method 800 can next include releasing the coiled implant 102 in the aneurysm sac A at step 804. Method 800 can finally include achieving, by the coiled implant 102, an increase in packing density in the aneurysm sac A compared to a comparative device having an approximately equal coil length of the coiled implant at step 806.

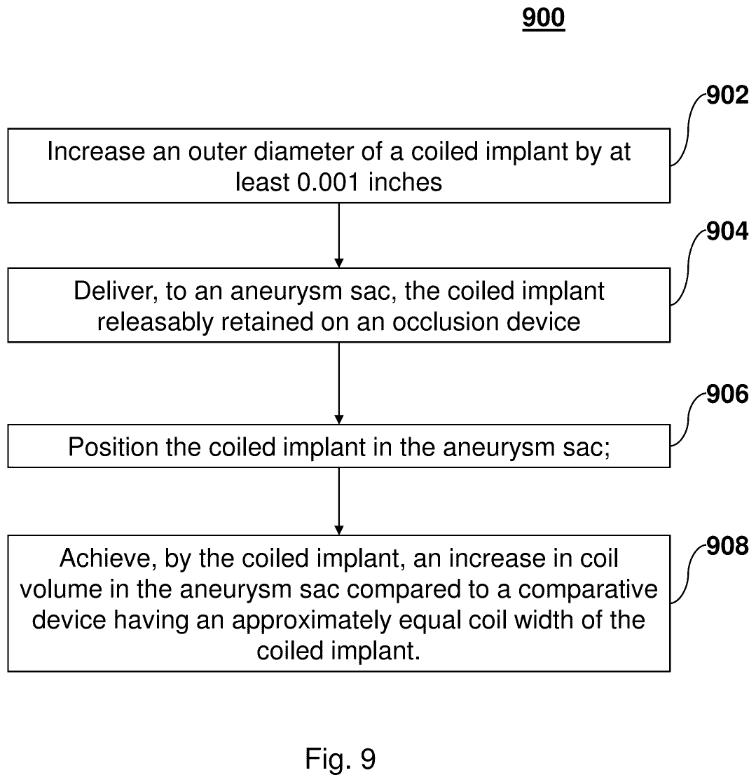

FIG. 9 is a flowchart of a method 900 of treating an aneurism. Prior to delivery of a coiled implant 102 to the aneurysm sac A, method 900 can include increasing an outer diameter D2 of a coiled implant 102 by at least 0.001 inches at step 902. Method 900 can then include delivering, to an aneurysm sac, a coiled implant 102 releasably retained on an occlusion device 100 at step 904. Method 900 can next include positioning the coiled implant 102 in the aneurysm sac A at step 906. Method 900 can finally include achieving, by the coiled implant 102, an increase in coil volume in the aneurysm sac A compared to a comparative device having an approximately equal coil width of the coiled implant at step 908.

The descriptions contained herein are examples of embodiments of the invention and are not intended to limit the scope of the invention. As described herein, the invention contemplates many variations and modifications of an occlusion device and methods of treating an implant, including alternative mechanical constructions, alternative geometries, alternative material selections, ancillary treatment steps, etc. Modifications and variations apparent to those having skilled in the pertinent art according to the teachings of this disclosure are intended to be within the scope of the claims which follow.

The following clauses list non-limiting embodiments of the disclosure:

Clause 1: A method for treating an aneurysm, the method comprising: delivering, to an aneurysm sac, an occlusion device comprising a coiled implant releasably retained on the occlusion device; releasing the coiled implant in the aneurysm sac; and achieving, by the coiled implant, an increase in packing density in the aneurysm sac compared to a comparative device having an approximately equal coil length of the coiled implant.

Clause 2: The method of clause 1, wherein the coiled implant further comprises an increased implant outer diameter of at least 0.001 inches compared to a comparative device having an approximately equal coil length of the coiled implant.

Clause 3: The method of clauses 1 or 2, further comprising increasing, by the coiled implant, a coil volume in the aneurysm sac by at least approximately 7%.

Clause 4: The method of any of clauses 1-3, wherein the coiled implant comprises a primary wind diameter ranging from about 0.008 inches to about 0.018 inches.

Clause 5: The method of any of clauses 1-4, wherein the coiled implant comprises an implant coil length of about 40 cm and an implant outer diameter of about 0.015 inches, and wherein the coiled implant increases the packing density in the aneurysm sac by increasing a coil volume by at least approximately 15% compared to a first comparative device comprising a first coil length of about 40 cm and a first outer diameter of about 0.014 inches.

Clause 6: The method of any of clauses 1-4, wherein the coiled implant comprises an implant coil length of about 6 cm and an implant outer diameter of about 0.012 inches, and wherein the coiled implant increases the packing density in the aneurysm sac by increasing a coil volume by at least approximately 44% compared to a second comparative device comprising a second coil length of about 6 cm and a second outer diameter of about 0.010 inches.

Clause 7: The method of any of clauses 1-4, wherein the coiled implant comprises an implant coil length of about 45 cm and an implant outer diameter of about 0.015 inches, and wherein the coiled implant increases the packing density in the aneurysm sac by increasing a coil volume by at least approximately 7% compared to a third comparative device comprising a third coil length of about 45 cm and a third outer diameter of about 0.014 inches.

Clause 8: The method of any of clauses 1-4, wherein the coiled implant comprises an implant coil length of about 20 cm and an implant outer diameter of about 0.012 inches, and wherein the coiled implant increases the packing density in the aneurysm sac by increasing a coil volume by at least approximately 8% compared to a fourth comparative device comprising a fourth coil length of about 20 cm and a fourth outer diameter of about 0.010 inches.

Clause 9: The method of any of clauses 1-4, further comprising: reducing a number of coiled implants delivered to the aneurysm sac compared to a number of comparative devices, wherein the comparative devices comprise an approximately equal coil length of the coiled implant.

Clause 10: A method for treating an aneurysm, the method comprising: delivering, to an aneurysm sac, a coiled implant releasably retained on an occlusion device; positioning the coiled implant in the aneurysm sac; and achieving, by the coiled implant, an increase in coil volume in the aneurysm sac compared to a comparative device having an approximately equal coil width of the coiled implant.

Clause 11: The method of clause 10, wherein the coiled implant further comprises an increased implant outer diameter of at least 0.001 inches compared to a comparative device having an approximately equal coil width of the coiled implant.

Clause 12: The method of clause 11, further comprising increasing, by the coiled implant, a coil volume in the aneurysm sac by at least approximately 7%.

Clause 13: The method of any of clauses 10-12, wherein the coiled implant comprises a primary wind diameter ranging from about 0.008 inches to about 0.018 inches.

Clause 14: The method of any of clauses 10-13, wherein the coiled implant comprises an implant coil length of about 42 cm, an implant coil width of about 12 mm, and an implant outer diameter of about 0.015 inches, and wherein the coiled implant increases the coil volume in the aneurysm sac by at least approximately 7% compared to a fifth comparative device comprising a fifth coil length of about 45 cm, a fifth coil width of about 12 mm, and a fifth outer diameter of about 0.014 inches.

Clause 15: The method of any of clauses 10-13, wherein the coiled implant comprises an implant coil length of about 15 cm, an implant coil width of about 5 mm, and an implant outer diameter of about 0.015 inches, and wherein the coiled implant increases the coil volume in the aneurysm sac by at least approximately 8% compared to a sixth comparative device comprising a sixth coil length of about 20 cm, a sixth coil width of about 5 mm, and a sixth outer diameter of about 0.010 inches.

Clause 16: The method of any of clauses 10-13, further comprising: reducing a number of coiled implants delivered to the aneurysm sac compared to a number of comparative devices, wherein the comparative devices comprise an approximately equal coil length of the coiled implant.

What is claimed is:

1. A method for treating an aneurysm, the method comprising:
delivering, to an aneurysm sac, an occlusion device comprising a coiled implant releasably retained on the occlusion device;
releasing the coiled implant in the aneurysm sac; and
achieving, by the coiled implant, an increase in packing density in the aneurysm sac compared to a comparative device having an approximately equal coil length of the coiled implant.

2. The method of claim 1, wherein the coiled implant further comprises an increased implant outer diameter of at least 0.001 inches compared to a comparative device having an approximately equal coil length of the coiled implant.

3. The method of claim 2, further comprising increasing, by the coiled implant, a coil volume in the aneurysm sac by at least approximately 7%.

4. The method of claim 1, wherein the coiled implant comprises a primary wind diameter ranging from about 0.008 inches to about 0.018 inches.

5. The method of claim 1, wherein the coiled implant comprises an implant coil length of about 40 cm and an implant outer diameter of about 0.015 inches, and wherein the coiled implant increases the packing density in the aneurysm sac by increasing a coil volume by at least approximately 15% compared to a first comparative device comprising a first coil length of about 40 cm and a first outer diameter of about 0.014 inches.

6. The method of claim 1, wherein the coiled implant comprises an implant coil length of about 6 cm and an implant outer diameter of about 0.012 inches, and wherein the coiled implant increases the packing density in the aneurysm sac by increasing a coil volume by at least approximately 44% compared to a second comparative device comprising a second coil length of about 6 cm and a second outer diameter of about 0.010 inches.

7. The method of claim 1, wherein the coiled implant comprises an implant coil length of about 45 cm and an implant outer diameter of about 0.015 inches, and wherein the coiled implant increases the packing density in the aneurysm sac by increasing a coil volume by at least approximately 7% compared to a third comparative device comprising a third coil length of about 45 cm and a third outer diameter of about 0.014 inches.

8. The method of claim 1, wherein the coiled implant comprises an implant coil length of about 20 cm and an implant outer diameter of about 0.012 inches, and wherein the coiled implant increases the packing density in the aneurysm sac by increasing a coil volume by at least approximately 8% compared to a fourth comparative device comprising a fourth coil length of about 20 cm and a fourth outer diameter of about 0.010 inches.

9. The method of claim 1, further comprising:
reducing a number of coiled implants delivered to the aneurysm sac compared to a number of comparative devices, wherein the comparative devices comprise an approximately equal coil length of the coiled implant.

10. A method for treating an aneurysm, the method comprising:
delivering, to an aneurysm sac, a coiled implant releasably retained on an occlusion device;
positioning the coiled implant in the aneurysm sac; and
achieving, by the coiled implant, an increase in coil volume in the aneurysm sac compared to a comparative device having an approximately equal coil width of the coiled implant.

11. The method of claim 10, wherein the coiled implant further comprises an increased implant outer diameter of at least 0.001 inches compared to a comparative device having an approximately equal coil width of the coiled implant.

12. The method of claim 11, further comprising increasing, by the coiled implant, a coil volume in the aneurysm sac by at least approximately 7%.

13. The method of claim 10, wherein the coiled implant comprises a primary wind diameter ranging from about 0.008 inches to about 0.018 inches.

14. The method of claim 10, wherein the coiled implant comprises an implant coil length of about 42 cm, an implant coil width of about 12 mm, and an implant outer diameter of about 0.015 inches, and wherein the coiled implant increases the coil volume in the aneurysm sac by at least approximately 7% compared to a fifth comparative device comprising a fifth coil length of about 45 cm, a fifth coil width of about 12 mm, and a fifth outer diameter of about 0.014 inches.

15. The method of claim 10, wherein the coiled implant comprises an implant coil length of about 15 cm, an implant coil width of about 5 mm, and an implant outer diameter of about 0.015 inches, and wherein the coiled implant increases the coil volume in the aneurysm sac by at least approximately 8% compared to a sixth comparative device comprising a sixth coil length of about 20 cm, a sixth coil width of about 5 mm, and a sixth outer diameter of about 0.010 inches.

16. The method of claim 10, further comprising:
reducing a number of coiled implants delivered to the aneurysm sac compared to a number of comparative devices, wherein the comparative devices comprise an approximately equal coil length of the coiled implant.
